(19) United States
(12) Patent Application Publication
(10) Pub. No.: US 2012/0308653 A1
(43) Pub. Date: Dec. 6, 2012

(54) WORMWOOD PILL CONTAINING 95% OF WORMWOOD ETHYL ALCOHOL EXTRACT THAT HAS INACTIVATION EFFICACY OF THE H1N1 VIRUS AND THE H9N2 AVIAN INFLUENZA VIRUS

(76) Inventors: Bongkwan Jo, Busan (KR); Heeyoung Shim, Busan (KR)

(21) Appl. No.: 13/577,106

(22) PCT Filed: Jan. 31, 2011
(86) PCT No.: PCT/KR11/00633

§ 371 Date: Aug. 3, 2012

(30) Foreign Application Priority Data
Feb. 3, 2010 (KR) 10-2010-0009824

(51) Int. Cl. A61K 36/282; A61K 9/36; A61K 9/48; A61P 31/16

(52) U.S. Cl. 424/451; 424/740; 424/479

(57) ABSTRACT

A mugwort pill containing a 95% ethanol extract of wormwood having an inactivation power with respect to a novel swine-origin influenza virus and an avian influenza virus is disclosed. In more details, the conventional mugwort pill is prepared by a hot water extraction method and has 0.8% of crude fat component; however the ethanol extract mugwort crude fat of the present invention is 11.6%, in particular the crude fat component is 14.5 times higher, and the novel swine-origin influenza virus (H1N1) can be inactivated 99.99%, and it has a good disinfection effect to an avian influenza virus (H9N2). When a mugwort pill containing a 95% ethanol extract of wormwood having an inactivation power with respect to a novel swine-origin influenza virus and an avian influenza virus is administrated to people or fowls, the people or fowls can have immunity with respect to a novel swine-origin influenza virus and an avian influenza virus.

### TECHNICAL FIELD

The present invention relates to a mugwort pill containing a 95% ethanol extract of wormwood having an inactivation power with respect to a novel swine-origin influenza virus and an avian influenza virus, and in particular to a mugwort pill which is characterized in that in case of the verification on the inactivation power with respect to a novel swine-origin influenza virus and an avian influenza virus, the inactivation power of a wormwood extract by means of a conventional herbal decoction extract is not recognized; however a constant inactivation function of a 95% ethanol extract of wormwood is verified at the same dilution ratio in the present invention.

### BACKGROUND ART

The mugwort pill is conventionally directed to a mugwort pill made from wormwood so as to help enhance the functions of a spleen and a stomach, and as another use of it, an Artemisia messerschmidtiana pill is generally used for the purpose of enhancing the function of liver. The above mentioned mugwort pill is generally made by boiling wormwood by means of a herbal decoction method; however it is impossible to extract essential oil components from the wormwood.

In order to extract the essential oil components from wormwood, there is an organic solvent extraction method. There was not any trial to produce a 95% ethanol extract of wormwood having an inactivation power with respect to a novel swine-origin influenza virus and an avian influenza virus so as to enhance the level of immunity of a human and fowl in such a way to extract a mugwort pill from a 95% ethanol alcohol.

### DISCLOSURE OF INVENTION

Accordingly, the inactivation power is compared and verified with respect to a novel swine-origin influenza virus and an avian influenza virus between a conventional mugwort pill and a mugwort pill made of a 95% ethanol extract of wormwood of the present invention.

To achieve the above objects, a research on the verification of an inactivation power with respect to a novel swine-origin influenza virus was conducted by the influenza virus research center of the veterinary college of Chungnam national university. In addition, a research on the disinfection effects with respect to an avian influenza virus was conducted by the veterinary college of Kyungpook national university, and a component analysis of a conventional mugwort pill and a mugwort pill made of a 95% ethanol extract of the present invention was conducted by the food analysis center of Pukyong national university.

### ADVANTAGEOUS EFFECTS

The present invention is directed to a mugwort pill containing a 95% ethanol extract of wormwood having an inactivation power with respect to a novel swine-origin influenza virus and an avian influenza virus. It was shown that the mugwort made of a 95% ethanol extract of wormwood had a crude fat which is 14.5 folds as compared with a mugwort pill made by a conventional herbal decoction method. Since the crude fat component of wormwood has an inactivation power with respect to a novel swine-origin influenza virus and an avian influenza virus, when a human takes it, the human can have immunity with respect to an avian influenza virus. When it is added to feed of fowl, it is possible to have enhanced immunity effects with respect to an avian influenza virus. In addition, the mugwort pill made of a 95% ethanol extract according to the present invention has ash which is reduced 70% as compare with the mugwort pill made by a conventional herbal decoction method, so one time dosage of mugwort pill decreases, which results in an easier administration. In other words, the mugwort pill made by a conventional herbal decoction method needs 20 through 30 pills for one time administration; however the mugwort pill made of a 95% ethanol extraction according to the present invention needs 1 through 3 pills for one time administration, which is a small amount of administration and an easier administration. In addition, the mugwort pill made of a 95% ethanol extract according to the present invention has a moisture component which decreased 44% as compared with the mugwort pill made by a conventional herbal decoction method, which helps lower the occurrence ratio of bacteria and enhance the safety of mugwort pills.

### BEST MODES FOR CARRYING OUT THE INVENTION

The following shows an experiment of an inactivation power with respect to a novel swine-origin influenza virus (H1N1) of a 95% ethanol extract of wormwood and a result of it.

### Experiment 1

### Experiment of Inactivation Power on a Novel Swine-Origin Influenza Virus (H1 N1) of Wormwood Extract

### 1. Purpose and Background of Experiment

The subject experiment is directed to analyzing an inactivation power with respect to a novel swine-origin influenza virus (H1N1) of mugwort

(Artemisia princeps or wormwood) requested from Chunyeonjiae corporation (CEO: Processor, J O, Bongkwan of Pukyong national university). The contracted research was conducted by the influenza virus research center of the veterinary college of Chungnam national university (Chief of the research center: Professor, Seo, Sang-hee), and after the experiment, the following result was obtained.

As an experiment background, the novel swine-origin influenza virus (H1N1), which came from swine, began to infect between humans in Mexico and the USA at the beginning of April, 2009 and spread throughout the world. World Health Organization announced a big infection in that year, and the death toll reached 10,000 persons. There is not a perfect cure medicine and vaccine to be prepared. The inactivation verification on the novel swine-origin influenza virus (H1N1) with a mugwort extract would be greatly helpful to an individual sanitation.

### 2. Samples and Method

Samples: The samples were came from a sample mugwort (Artemisia princeps or wormwood) requested from Chunyeonjiae corporation. The samples were processed via the steps of preparing a wormwood extract in order to verify the inactivation power on the novel swine-origin influenza virus (H1N1), which steps comprise a step of grinding dried leaves of Artemisia princeps or wormwood, a step of precipitating it in 95% alcohol, 95% ethanol for 24 to 48 ours, a step of enough extracting a pharmacological component of mugwort in alcohol 95% ethanol, a step of separating mugwort leaves and impurities, and a step of vaporizing alcohol 95% ethanol for thereby finally producing a mugwort extract. An emulsifier of the same amount based on that of a mugwort extract is added, and it is finally diluted with a distilled water of the 98 weight folds.

Method: as a virus to be used for the experiment on the mugwort extract, a novel swine-origin influenza virus (H1 N1), on which a big spread was warned, was used, and it was a WHO standard strain (A/California/04/09 (H1N1), and as a measuring method, 1) 100 μl of the novel swine-origin influenza virus (H1N1) 10⁶EID₅₀/(million, the amount corresponding to 50% egg infection) was applied to the mugwort extract 100 μl ) It was reacted for 20 minutes in the experiment room temperature. 3) It was reacted for 20 minutes and then was consecutively diluted with PBS by 10 folds. 4) The liquid diluted by 10 folds was inoculated to an embryonated chicken egg of the age of 10 days. 5) The inoculated, embryonated chicken egg was grown in an incubator of 35 for 48 hours, and was stored in a refrigerator 4 for 4 hours. 6) The presence of virus was checked at the lumbar region of a spinal cord of the cool-stored and embryonated chicken egg by means of a hemagglutination, thus measuring log 10 egg infectious dose 50/ml (log₁₀EID₅₀).

### 3. Result of the Experiment on Inactivation Power

TABLE 1
Table 1. Result sample of inactivation power of a novel
swine-origin influenza virus (H1N1) of mugwort extract

| | Virus titer |
|---|---|
| Samples | [log₁₀EID₅₀/ml] |
| Mugwort (Artemisia princeps or wormwood) | <1.0, <1.0, <1.0, <1.0, <1.0 |
| extract liquid | |
| Virus comparison group | 6.0, 6.5, 6.0, 6.0, 6.0 |

Like the result of the inactivation power of the novel swine-origin influenza virus (H1 N1) of the mugwort extract liquid in the table 1, the mugwort (Artemisia princeps or wormwood) of Chunyeonjiae corporation has confirmed that inactivates the novel swine-origin influenza virus (H1 N1) up to 99.9%.

The following shows an experiment and a result of the disinfection effects of the wormwood extracts with respect to the avian influenza virus (H1N1).

### Experiment Example 2

Disinfection Effects of Wormwood Extracts with Respect to the Avian Influenza Virus (H9N2)

### Experiment

### 1. Samples and Pathogen Used in Test

Using three kinds of B, C and D of the mugwort (Artemisia princeps or wormwood) extracts requested from Chunyeonjiae corporation as the experiment materials, the disinfection effects were analyzed. A result was obtained after the veterinary college of Kyungpook national university conducted a contracted service research.

Samples: The mugwort extract B was processed a step of grinding dried leaves of Artemisia princeps or wormwood, a step of precipitating it in 95% alcohol, 95% ethanol for 24 to 48 ours, a step of enough extracting a pharmacological component of mugwort in alcohol 95% ethanol, a step of separating mugwort leaves and impurities, and a step of vaporizing alcohol 95% ethanol for thereby finally producing a mugwort extract. An emulsifier of the same amount based on that of a mugwort extract was added for thereby producing a raw material. The mugwort extract C was made of a mugwort extract liquid extracted in a half-solidified state in the boiling water like grain syrup. The mugwort extract D was provided in the same method as the method of producing wormwood extract in the production process of the mugwort extract D except for the addition of the emulsifier in the same amount.

The researched and used pathogen was an avian influenza virus A/chicken/Korea

MS96/1996(H9N2), and three kinds B, C and D of the mugwort extract raw liquid were diluted with the dilution folds of 100 folds, 200 folds, 400 folds and 800 folds and were used, and the experiment method was performed in compliance with the disinfection agent effectiveness experiment standard of the virus of the appendix table 2 of the disinfection agent effectiveness experiment guideline of Notification number 2007-10 of National Veterinary Research and Quarantine Service.

### 2. Notified Virus

1) Virus: in order to evaluate the disinfection effectiveness with respect to the virus of the notified disinfection agent, the avian influenza virus H9N2 (MS96 strain), which were assigned from National Veterinary Research and Quarantine Service and were stored, was used in the experiment.
2) Virus cultivation: the avian influenza virus MS96 strain was cultivated in the SPF incubator of the age of 9 days, and they were cultivated for the titer of the virus to be over minimum 10^{7.0}ED₅₀, and the cultivated virus were stored in the freezer which maintained at below zero temperature of 70 degrees and were used.

### 3. Experiment Method

1) Dilution of disinfection agent: The dilution of each notified disinfection agent was conducted with an organic dilution liquid (5% cow fetus blood serum, and the dilution folds wee 1/100, 1/200, 1/400 and 1/800.
2) Reaction of disinfection agent: the cultivated virus liquid (allantoic fluid) 1.0 of 4 were respectively mixed with the organic dilution liquid (4% cow fetus blood serum) 19 based on the treatment structure, so the virus for a disinfection gent reaction was prepared, and 2.5 of the liquid of the virus prepared every minute was inputted into the test tube with a disinfection dilution liquid of the same amount of state 4 (total 5), and they were reacted accurately for 30 minutes and swung every 10 minutes during the reaction, and light water is used for the comparison group instead of disinfection liquid.
3) Neutralization reaction: After the reaction of disinfection agent was completed, 1.0 was immediately taken out for neutralizing the effectiveness of the disinfection agent and was mixed and neutralized with PBS of 37 containing the non-equalized 10% cow fetus blood serum of 10% of the same amount.

Growth judgments of virus: The neutralization liquid was diluted to raw liquid, 10⁻¹, 10⁻², 10⁻³, 10⁻⁴, and 10⁻⁵ respectively by using the PBS, and 0.2 was cultivated in allantoic fluid of the grown egg of the age of 10 days of five eggs per dilution fold and was cultivated in the incubator of 37. The eggs were checked every day, and the grown eggs died within 24 hours after inoculation were judged dead and were excluded from the experiment performance.
5) Disinfection agent effective concentration evaluation: The dilution concentration of which the inactivation of over 10^{4.0}EID₅₀ was recognized was considered as effective concentration with the amount of the virus of the disinfection agent treatment group as a comparison in the amount of the virus of the pathogen comparison group.

At this time, the quantity of the virus was counted using the Karber method.

### Result of the Experiment

### Table 2

Disinfection effectiveness experiment test material based on disinfection agent dilution folds with respect to the avian influenza virus of mugwort extract of Table 2.

| | Dilution | Neutralation liquid dilution folds(number of growths/total | | | | | | | |
|---|---|---|---|---|---|---|---|---|---|
| | folds of | number of inoculation) | | | | | | Virus titer and decrease | |
| test | disinfection | raw | | | | | | level | |
| materials | agent | liquid | 10⁻¹ | 10⁻² | 10⁻³ | 10⁻⁴ | 10⁻⁵ | titer(EID₅₀₎ | decrease(1og₁₀₎ |

| | | | | | | | | | |
|---|---|---|---|---|---|---|---|---|---|
| B | 1/1001/200 | 0/45/5 | 0/55/5 | 0/43/ | 0/52/53/54/5 | 0/50/30/52/5 | 0/52/50/51/4 | 010^{2.9}10^{3.2}10^{3.9} | 5.12.22.01.2 |
| | 1/4001/800 | 5/55/5 | 5/55/5 | 55/55 | | | | | |
| | | | | /5 | | | | | |
| C | 1/1001/200 | 5/55/5 | 5/55/5 | 5/55/ | 3/54/54/55/5 | 2/54/53/43/4 | 1/50/51/50/5 | 10^{3.7}10^{3.7}10^{4.1} | 1.41.41.01.0 |
| | 1/4001/800 | 5/55/5 | 5/55/5 | 55/55 | | | | 10^{4.1} | |
| | | | | /5 | | | | | |
| D | 1/100 | 0/50/5 | 0/50/5 | 0/50/ | 0/50/55/55/5 | 0/50/52/55/5 | 0/50/50/51/5 | 0010^{3.9}10^{4.7} | 5.1 5.11.20.4 |
| | 1/2001/400 | 5/55/5 | 5/55/5 | 55/55 | | | | | |
| | 1/800 | | | /5 | | | | | |

| | | | | | | | |
|---|---|---|---|---|---|---|---|
| Pathogen comparison | 5/5 | 5/5 | 5/5 | 5/5 | 5/5 | 3/5 | 10^{5.0} |

As seen in the result of the disinfection effectiveness test based on the disinfection agent dilution folds with respect to the avian influenza virus of the mugwort extract of Table 2, in case of the agent B as a result of the test on the disinfection effects with respect to the avian influenza virus by the agents of the mugwort extract, the disinfection effects were recognized in case of the 100 folds ( 1/100) dilution liquid of the raw liquid by the mugwort extract agents, and in case of the agent C, the disinfection effects were not recognized in case of the 100 folds ( 1/100) dilution liquid of the raw material, and in case of the agent D, the disinfection effects were recognized until the 200 folds ( 1/200) dilution concentration of the raw liquid.

As a result of the above mentioned experiment, in case of the wormwood extract, the inactivation of the novel swine-origin influenza (H1N1) virus (Table 1) and the disinfection effects (Table 2) with respect to the novel swine-origin influenza virus were recognized; however the mugwort pill by means of the conventional wormwood hot water extraction method was not recognized for the disinfection effects with respect to the novel swine-origin influenza virus. In order to study the reasons of it, the analysis of the conventional herbal decoction extraction mugwort pills and the key components of the 95% ethanol extraction mugwort pills of the present invention were requested to the food analysis center of Pukyong national university.

### Experiment 3

### Component Analysis of 95% Ethanol Extraction Mugwort Pills of the Present Invention and the Conventional Hot Water Extraction Mugwort Pills

Test performance and test items of 95% ethanol extract mugwort pills of the present invention, and conventional herbal decoction extraction mugwort pills in Table 3.

| | 95% ethanol extraction | Herbal decoction extraction |
|---|---|---|
| test items | mugwort pills | mugwort pills |
| Calorie | 376.0 Kcal/100 g | 280.0 Kcal/100 g |
| Carbohydrate | 70.2 g | 67.8 g |
| Crude protein | 11.6% | 12.8% |
| Crude fat | 11.6% | 10.8% |
| Moisture | 2.2% | 3.9% |
| Ash | 4.4% | 14.7% |
| Number of bacteria | 6.0 × 10³ cfu/g | 9.0 × 10⁴ cfu/g |

As seen in Table 3, the 95% ethanol extraction mugwort pills and the herbal decoction extraction mugwort pills were selected as samples when they had similar material components and component ratios. As a result, the 95% ethanol extract mugwort pills of the present invention were characterized to have the crude fat component 14.5 folds higher as compared with the conventional herbal decoction extraction mugwort pills. Since the crude fat components of the wormwood has an essential oil component such as cineol, alpha-thujone for thereby providing a unique pharmacological action. In other words, the 95% ethanol extract mugwort pills according to the present invention has an inactivation power with respect to a novel swine-origin influenza virus and an avian influenza virus, and it is expected to enhance the immunity when it is administrated to people or fowl. In addition, the ash in the 95% ethanol extraction mugwort pills of the present invention has decreased 70% as compared with the conventional hot water extraction mugwort pills, which helps people intake a small amount of one dosage of the mugwort pills. In addition, the moisture component of the 95% ethanol extraction mugwort pills of the present invention has decreased 44% as compared with the conventional herbal decoction extraction mugwort pills, which helps decrease the occurrence of bacteria and helps enhance the stability of mugwort pills.

### MODES FOR CARRYING OUT THE INVENTION

The present invention is directed to a mugwort pills containing 95% ethanol extract of wormwood having an inactivation power with respect to a novel swine-origin influenza virus and an avian influenza virus. In more details, there is provided a step for preparing a wormwood extract. The mugwort extract is prepared in such a way that dried leaves of Artemisia princeps or wormwood are ground using a grinder, and are precipitated in 95% alcohol 95 ethanol for 24 to 48 hours, and the pharmacological components are enough extracted into alcohol 95% ethanol, and the leaves of mugwort and impurities are removed, and alcohol 95% ethanol is vaporized, thus producing wormwood 95% ethanol extract.

Hereinafter the construction of the present invention will be described in details by way of the following embodiments, and the scope of the present invention is not limited to the disclosed embodiments, and various modifications are possible within the scope without parting from the scope and concept of the present invention.

The present invention will be described by way of the following embodiments.

### Embodiment 1

### Mugwort Pills Containing Wormwood 95% Ethanol Extraction

The mugwort pills of the present invention is characterized to contain 10˜90% of the wormwood 95% ethanol extract. In particular, when it is administrated to people or fowls, it is possible to adjust the amount of it. For example, for people the composition ratio of the wormwood 95% ethanol extract can be increased, and for fowls, the composition ratio of the wormwood 95% ethanol extract can be decreased. According to “Artemisia princeps” written by Mae Jun Hee (Chinese medical publication company, Beijing, January 1999), it says reciting “Chinese medicine handbook” that wormwood is nontoxic, and if it is administrated for a long time, side effects such heat collision, hyperemia, etc. might occur. So, according to a normal intake standard of wormwood, it is recommended to intake 3˜9 g in its dosage per day in terms of a decoction form, and it is 1˜3 g in case of a pill form. According to the wormwood gasoline dangeupyun, 450˜600 mg is recommended. Since the wormwood 95% ethanol extract pills according to the present invention does not have toxic, it is possible to make the wormwood 95% ethanol extract composition be maximum 90%, and the rest portions of 10% may be made in the form of mugwort pills hardened using the powder of malt. In the embodiment 1, the wormwood pills are prepared with 70% of wormwood 95% ethanol extract, 10% of black garlic, 4% of Korean angelica, 3% of atractylodes macrocephala Koidzumi, 4% of date, and 10% of malt in the scope of compositions.

### Embodiment 2

Mugwort Pills Containing Wormwood 95% Ethanol Extracts the Surface of which is Processed with a Sugar Coated Tablet

The mugwort pills of the embodiment 1 are inputted into the sugar coating machine, and a honey water mixed with honey and water at a ratio of 1:1 is uniformly sprayed on the mugwort pills, and the mugwort pills are prepared with the wormwood 95% ethanol extract the surface of which is processed with a sugar coating tablet in a sugar coating machine. The embodiment 2 is directed to using honey as an example among multiple materials used to make the surface of the mugwort pill have a taste.

### Embodiment 3

Mugwort Pills Containing Wormwood 95% Ethanol Extract which is Formed in a Swallow Type Capsule.

The mugwort pills of the embodiment 1 are made in the form of powder in the grinding machine and are inputted in a swallow type capsule for thereby preparing the wormwood ethanol extract mugwort pills made in the form of a capsule. At this time, when preparing the mugwort pills of the embodiment 1 in the form of powder, it is needed to lower the viscosity of the mugwort pills. The ratio of the wormwood extract is lowered down to 10%, and the ratio of powder of malt is increased instead. The embodiment 3 is directed to an example of the swallow type capsule among various methods in terms of treating the capsule for the purpose of swallowing mugwort pills.

Mugwort pills containing wormwood 95% ethanol extract which is converted into a liquid pouch by mixing with water

The mugwort pills of the embodiment 1 are inputted into the grinder and are made in the form of powder. It is mixed with water and boiled for thereby preparing a liquid type ethanol extract the phase of which is changed to a liquid phase. The prepared liquid type ethanol extract is inputted into the pouch and is swallowed by a user. The embodiment 4 is directed to an example in which the wormwood 95% ethanol extract is not formed in the form of pills, but is formed in the form of a liquid phase.

### INDUSTRIAL APPLICABILITY

It is characterized in that the mugwort pills containing a wormwood 95% ethanol extract according to the present invention has an inactivation power with respect to a novel swine-origin influenza virus and an avian influenza virus, and when it is administrated to people or fowls, there is an effect of enhancing immunity.

### Certificate of Test

Verification of inactivation on novel swine-origin influenza virus (H1N1) of Chunyeonjiae corporation's mugwort (Artemisia princeps or wormwood) extract (name of product: Rtemi)

Conclusion: It is confirmed that Chunyeonjiae corporation's mugwort (Artemisia princeps or wormwood) extract liquid kills 99.99% of novel swine-origin influenza (H1N1) virus.
Requester of research: Chunyeonjiae corporation
Address: 2F, 881-12, Daeyeon 1dong, Nam-gu, Busan city
CEO: J O Bongkwan (professor of Pukyong national university)
Head official of influenza virus research center
Veterinary college of Chungnam national university
Person in charge: Professor Seo Sang-hee
Dec. 30, 2009
Head official of School-industry cooperation center of Chungnam national university

### Confirmation Sheet of Test

Verification on inactivation of a novel swine-origin influenza virus (H1 N1) of the product name of Rtemi of Chunyeonjiae corporation's mugwort (Artemisia princeps or wormwood) extract
Head official of influenza virus research center
Veterinary college of Chungnam national university
Person in charge: Professor Seo Sang-hee
December 2009
Head official of School-industry cooperation center of Chungnam national university

### 1. Purpose of Test

It is aimed to analyze the inactivation power of novel swine-origin influenza virus (H1N1) of the mugwort (artemisia princeps or wormwood as Chunyeonjiae corporation (CEO: Processor, JO Bongkwan of Pukyong national university) requests.

### 2. Background of Test

The novel swine-origin influenza virus (H1N1), which came from swine, began to infect between humans in Mexico and the USA at the beginning of April 2009 and spread throughout the world. World Health Organization announced a big infection in that year, and the death toll reached 10,000 persons. There is not a perfect cure medicine and vaccine to be prepared. The inactivation verification on the novel swine-origin influenza virus (H1N1) with a mugwort extract would be greatly helpful to an individual sanitation.

### 3. Sample of Measurements

The samples [mugwort (Artemisia princeps or wormwood) the analysis of which is requested from Chunyeonjiae corporation are used.

### 4. Method of Measurements

4.1 Virus to be researched: it is a novel swine-origin influenza virus (H1N1) the spread of which was warned, and is a WHO standard strain (A/California/04/09 (H1N1).

### 4.2 Method of Measurements

1. 100 μl of the novel swine-origin influenza virus (H1N1) 10⁶EID₅₀/ml (million, the amount corresponding to 50% egg infection) was applied to the mugwort extract liquid(100 μl).
2. It is reacted at the temperature of the experiment room for 20 minutes.
3. After it is reacted for 20 minutes, it is continuously diluted with PBS by 10 folds.
5. The liquid diluted by 10 folds is inoculated to an embryonated chicken egg of the age of 10 days.
6. The inoculated, embryonated chicken egg was grown in an incubator of 35 for 48 hours, and was stored in a refrigerator 4 for 4 hours.
7. The presence of virus was checked at the lumbar region of a spinal cord of the cool-stored and embryonated chicken egg by means of a hemagglutination, thus measuring log 10 egg infectious dose 50/ml (log₁₀EID_{50/ml}).

### 5. Result of Test

| | Virus titer |
|---|---|
| Samples | [log₁₀EID₅₀/ml] |
| Mugwort (Artemisia princeps or wormwood) | <1.0, <1.0, <1.0, <1.0, <1.0 |
| extract liquid | |
| Virus comparison group | 6.0, 6.5, 6.0, 6.0 , 6.0 |

### CONCLUSION

It is confirmed that Chunyeonjiae corporation's mugwort (Artemisia princeps or wormwood) extract liquid inactivates 99.99% of novel swine-origin influenza (H1N1) virus.

School-industry cooperation of Chungnam national university Atten: CEO of Chunyeonjiae corporation
Via
Title: Submission of report on result of contracted searches
1. We hope that your company becomes prosperous.
2. It is noted that as attached we herewith submit a report (confirmation sheet of test) on a result of the following contracted research supported by your company and conducted by our university.
3. Contents of researches
Monetary unit: won

| Person in charge | | Title of | Organization | Cost of |
|---|---|---|---|---|
| Department | Name | research | of research | research |

| | | | |
|---|---|---|---|
| Veterinary | Seo Sang- | Verification on | Sep. 28, 2009~ |
| college | hee | inactivation of | Dec. 30, 2009 |
| | | novel swine- | |
| | | origin influenza | |
| | | virus (H1N1) | |
| | | of mugwort extract | |

Attached: A copy of a report of the result (Korean and English) End

Head official of School-industry cooperation center of Chungnam national university

1. A mugwort pill containing a 95% ethanol extract of wormwood, comprising:
10˜90% of a wormwood 95% ethanol extract to have an inactivation power with respect to a novel swine-origin influenza virus and an avian influenza virus.

2. A mugwort pill containing a 95% ethanol extract of wormwood according to claim 1, wherein the mugwort pill is surface-processed with a sugar coated tablet.

3. A mugwort pill containing a 95% ethanol extract of wormwood according to claim 1, wherein the mugwort pill is encased in a swallow type capsule.

4. A mugwort pill containing a 95% ethanol extract of wormwood according to claim 1, wherein the mugwort pill is made in a liquid phase form by mixing mugwort pill with water.
